(19) United States
(12) Patent Application Publication
(10) Pub. No.: US 2023/0277578 A1
(43) Pub. Date: Sep. 7, 2023

(54) ORAL DELIVERY OF NICOTINAMIDE ADENINE DINUCLEOTIDE (NAD) AND OTHER NUTRITIONAL SUPPLEMENTS

(71) Applicant: Craig KONIVER, Charleston, SC (US)

(72) Inventor: Craig KONIVER, Charleston, SC (US)

(21) Appl. No.: 18/010,324

(22) PCT Filed: Jun. 18, 2021
(86) PCT No.: PCT/US2021/038054

§ 371 Date: Dec. 14, 2022

Related U.S. Application Data
(60) Provisional application No. 63/040,994, filed on Jun. 18, 2020.
(60) Provisional application No. 63/140,776, filed on Jan. 22, 2021.

(51) Int. Cl. A61K 31/7084; A61K 45/06; A61K 31/00; A61K 9/68; A61K 9/00

(52) U.S. Cl. CPC A61K 31/7084; A61K 45/06; A61K 31/658; A61K 9/0058; A61K 9/0095

(57) ABSTRACT

A method of orally administering nicotinamide adenine dinucleotide (NAD) to an individual includes placing an oral delivery vehicle containing NAD into an individual's mouth for a delivery time to allow the NAD to absorb through the blood vessels in the mouth. The oral delivery vehicle may be configured for chewing or to slowly dissolve to increase delivery time, or may be a liquid beverage that is held in the mouth for a delivery time. Additional ameliorating ingredients may be included in the oral delivery vehicle that enhance absorption through the blood vessels or into the cell or provide an additional therapeutic effect. A stimulant such as caffeine may be included in a ratio of about 1:1 to the NAD concentration.

### CROSS-REFERENCE TO RELATED APPLICATIONS

This application claims the benefit of U.S. Provisional Application No. 63/040,994, filed on Jun. 18, 2020 and to U.S. Provisional Application No. 63/140,776 filed on Jan. 22, 2021; the entirety of both of which are incorporated herein by reference.

### BACKGROUND OF THE INVENTION

### Field of the Invention

This disclosure relates to a method of orally administering nicotinamide adenine dinucleotide (NAD) to an individual through the use of an oral delivery vehicles. Additionally, this disclosure relates to the delivery of caffeine and/or other substances with NAD. This disclosure also relates to the use of a beverage as an oral delivery vehicle for NAD and any accompanying nutritional supplements.

### Background

Nicotinamide adenine dinucleotide (NAD) is a co-enzyme that is key to cellular metabolism (e.g., chemical reactions in our cells, including chemical reactions that turn nutrients into energy, as well as chemical reactions that produce proteins and chemical reactions that eliminate waste). More specifically, NAD fuels basic functions of the mitochondria in cells. Biochemically, NAD+ is utilized by our mitochondria to produce ATP energy. For example, NAD receives and donates electrons (i.e., “electron transfer,” in which the oxidized form of NAD, which is referred to as “NAD+,” accepts electrons and its reduced form, which is referred to as “NADH,” donates electrons) in various chemical reactions that occur in the mitochondria. NAD also serves as a substrate of enzymes that add or remove chemical groups from other proteins. NAD also plays a key role in communicating between the nucleus of the cell and its mitochondria (i.e., cellular communication).

As we age, our bodies produce less NAD. When less NAD is available, the communication between the nuclei and the mitochondria in our cells is impaired, as is cellular metabolism. Thus, over time, decreasing levels of NAD impairs our cells' ability to make energy, which leads to aging and disease and may perhaps be a key factor in why our bodies age. Thus, NAD supplementation may address impairments to cellular communication and metabolism that occur with aging.

Conventionally, NAD supplementation has not been administered through common modes of administering nutritional supplements—pills (e.g., capsules, tablets, etc.) and drinks. Unlike its precursors nicotinamide riboside (NR) and nicotinamide mononucleotide (NMN), which are relatively small hydrophilic (water soluble) molecules, NAD is degraded in the gastrointestinal (GI) tract; for example, by the stomach and liver. As a result, NR and NMN supplements, which can survive conditions in the GI tract, are often taken in an effort to increase levels of NAD within cells. The synthesis of NAD from NR and NMN, however, consumes the cell's energy.

Although it is possible to bypass the GI tract and avoid the unnecessary consumption of energy within the cells by delivering NAD directly to the bloodstream (e.g., intravenously, by way of intraperitoneal injections, etc.), intravenous administration of NAD often costs over $1,000 a day and requires a patient to be hooked up to a drip IV for 8 hours.

### SUMMARY OF THE INVENTION

In various aspects, the delivery of nicotinamide adenine dinucleotide (NAD) in any suitable form (e.g., its oxidized form, NAD+, etc.) orally (e.g., sublingually, etc.) into a subject's bloodstream is disclosed. The term “oral” and its variants refer to the administration of a substance to a subject's mouth, from which the substance may be absorbed into the subject's bloodstream. The term “sublingual” and its variants refer to a type of oral administration of a substance to the blood vessels under a subject's tongue. Oral and sublingual delivery of NAD enable the NAD to enter into the subject's bloodstream before the delivery vehicle (e.g., a beverage, gum, soluble tablet, etc.) for the NAD enters the gastrointestinal tract, where the NAD might otherwise be digested or otherwise degraded and, thus, rendered ineffective; thus, oral and sublingual delivery provides a more direct path for the NAD from outside of the subject's body to the subject's cells, where the NAD can be readily utilized.

An exemplary oral delivery vehicle may comprise a beverage, throughout which the NAD may be dispersed. Other embodiments of oral delivery vehicles include tablets, such as a chewable tablet or chewing gum, a soluble tablet, drop, gels, and other liquid and semi-liquid compositions. An oral delivery vehicle may be a solid and may include ingredients to delay solubility, such as gelatin, mucilage, softeners/plasticizers, gum, such as gum Arabic and synthetic ingredients, such as polymers.

An oral delivery vehicle may include an effective concentration of NAD+, such as about 10 mg or more, about 15 mg or more, about 20 mg or more, about 25 mg or more, about 50 mg or more, about 100 mg or more and any range between and including the values provided. The amount of NAD included in the oral delivery vehicle may be dependent on the size of the person it is administered, wherein a larger person may require a high dose to produce an effective increase in NAD concentration. Other personal factors may also be important, such as base level NAD, age, percent body fat and the like.

In addition to NAD, an exemplary oral delivery vehicle may include one or more ameliorating therapeutic ingredient. As an example, an oral delivery vehicle may include one or more components that will enhance a subject's energy and may aide in the absorption of the NAD+ into the oral blood vessels and/or into the cell. Without limitation, such an energy-enhancing component may comprise a stimulant, such as caffeine, ephedrine, Amphetamines, lisdexamfetamine, and methylphenidate.

The ratio of NAD to caffeine may be provided to enhance absorption and may be about 0.5:1 or more, about 0.75:1 or more, about 0.1:1 or more, about 1.25:1 or more, about 1.5:1 or more, about 2.0:1 or more and any range between and including the NAD to caffeine ratios provided. One of caffeine's main roles of execution biochemically is blocking adenosine receptors. Adenosine, when activated, instills feeling of calm and relaxation. Therefore, when adenosine receptors are blocked, one feels more alert and aroused. NAD is partially broken down into Adenosine itself. Therefore, combining caffeine and NAD allows for more adenosine to be produced (via NAD) and more adenosine to be blocked (via caffeine). Therefore, in order for these two nutrients to work their best, it may be desirable to keep the NAD/stimulant ratio to about 1:1. If the ratio is too far from 1:1, the oral delivery vehicle may not achieve the desired effect increased mental acuity, alert activity combined with sustained energy. Caffeine helps with energy by blocking adenosine receptors. Caffeine also works in the mitochondria increasing a molecule called PGC-1 alpha. PGC-1a is a transcriptional coactivator that regulates the genes involved in energy metabolism. It is the master regulator of mitochondrial biogenesis. NAD also increases PGC-1 alpha. As such, combining these nutrients in equal ratios allows for increases in PGC-1 alpha.

Other ameliorating therapeutic ingredients that may be included in the delivery vehicle include, vitamins, such as B vitamins, cannabidiol, electrolytes, amino acids and so-called “ketone salts.” B vitamins help convert dietary energy into adenosine triphosphate (ATP), the type of energy used by the body. Ketone salts, such as beta-hydroxybutyrate (βHB or BHB) salts, provide energy to the body and are believed to kick-start the ketogenic process. Any of the ameliorating therapeutic ingredients may be included in concentration to produce an effective result and may be about 10 mg or more, about 20 mg or more, about 50 mg or more, about 75 mg or more, about 100 mg or more and any range between and including the concentrations provided.

In a specific embodiment, a nutritional supplement may include an oral delivery vehicle that carries NAD+. The oral delivery vehicle may comprise a beverage. In some embodiments, the oral delivery vehicle may comprise a so-called “smoothie,” which may provide an individual with a variety of nutrients. In other embodiments, the oral delivery vehicle may comprise a so-called “sports drink,” which may be formulated to restore electrolytes that are lost during intense physical activity. A serving of the nutritional supplement (e.g., a serving of the beverage, a bottle of the beverage, etc.) may include 25 mg or more of the NAD. Such a nutritional supplement may optionally include an energy-enhancing component. The energy-enhancing component may include a stimulant (e.g., caffeine, etc.). More specifically, a dose of the nutritional supplement may include 25 mg or more of the stimulant.

Methods for delivering NAD+ are also disclosed. Such methods include delivering NAD to the blood vessels within a subject's mouth, including but not limited to blood vessels in the bottom of a subject's tongue, or oral administration. The NAD may be orally administered alone or with another substance, such as an energy-enhancing component or other optional ingredients. The energy-enhancing component may comprise a stimulant, such as caffeine. In some embodiments, a method for delivering NAD may include introducing the oral delivery vehicle carrying NAD into a mouth of a subject (e.g., an individual, etc.) and having the subject hold the oral delivery vehicle in the mouth for a delivery time, that is effective to enable at least some of the NAD to be absorbed into blood within the subject's mouth. The subject may hold the oral delivery vehicle in his or her mouth momentarily. For example, the subject may be instructed to resist the urge to swallow while the oral delivery vehicle is initially present within the subject's mouth (e.g., for a couple seconds or more, such as about 2 seconds or more, about 3 seconds or more, about 5 seconds or more, about 10 seconds or more, about 30 seconds or more, about one minute or more, about two minutes or more, about five minutes or more and any range between and including the delivery times provided. Some oral delivery vehicles may slowly release the NAD and therefore holding the oral delivery vehicle in their mouth for a longer duration, one minute or more, may increase the absorption of the NAD. Chewing gum and soluble gels may be configured for slow release of the NAD as well as other ingredients. Once the oral delivery vehicle is swallowed, more of the oral delivery vehicle may be introduced into the subject's mouth, held within the subject's mouth (e.g., momentarily, etc.), and then swallowed. This process may be repeated until the subject has consumed a serving of the oral delivery vehicle. Each time the oral delivery vehicle is held within the subject's mouth, at least some of the NAD and any additional ingredients may be absorbed into the subject's bloodstream and, thus, administered to the subject. Additional ingredients that are not absorbed into blood vessels in the subject's mouth and into the subject's bloodstream may be absorbed the subject's bloodstream through his or her gastrointestinal (GI) tract.

### EXAMPLE 1

A study was performed to measure the increase in NAD after placing an oral delivery vehicle comprising nicotinamide adenine dinucleotide in the mouth for a delivery time. Two individuals, an adult male and an adult female, were tested by having their blood drawn at a 11:30 am for a pre-NAD concentration evaluation. The two individuals then chewed on one piece of chewing gum containing 25 mg of NAD, 25 mg of caffeine and 10 mg of CBD. At 12:30 pm, one hour after the pre-NAD blood sample, a test blood sample was taken. All samples were sent to Jinfiniti Precision Medicine, Augusta Ga., for evaluation of NAD concentration. The results are provided in Table 1. Both subjects had an increased NAD level with the adult female having a 1.5 μM increase or 5.8% and the male with a 0.6 μM, or 2.3% increase. The oral delivery vehicle containing NAD had a positive increase on the NAD as determined by this study.

TABLE 1

| | Pre-NAD | Test NAD | | |
|---|---|---|---|---|
| | Concentration | Concentration | | |
| | (uM) | (uM) | Increase | % Increase |
| Adult Female | 24 | 25.4 | 1.4 | 5.8% |
| Adult Male | 26.4 | 27 | 0.6 | 2.3% |

Other aspects of this disclosure, as well as features and advantages of various aspects of this disclosure, should become apparent to those of ordinary skill in the art through consideration of the foregoing disclosure and the appended claims.

Although the preceding disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

### BRIEF DESCRIPTION OF SEVERAL VIEWS OF THE DRAWINGS

The accompanying drawings are included to provide a further understanding of the invention and are incorporated in and constitute a part of this specification, illustrate embodiments of the invention, and together with the description serve to explain the principles of the invention.

FIG. 1 shows a perspective view of an exemplary oral delivery vehicle that is a tablet in the form of a stick of gum.

FIG. 2 shows a perspective view of an exemplary oral delivery vehicle that is a soluble tablet and the dissolved tablet.

FIG. 3 shows a perspective view of an exemplary oral delivery vehicle that is a beverage in a beverage container.

Corresponding reference characters indicate corresponding parts throughout the several views of the figures. The figures represent an illustration of some of the embodiments of the present invention and are not to be construed as limiting the scope of the invention in any manner. Some of the figures may not show all of the features and components of the invention for ease of illustration, but it is to understood that where possible, features and components from one figure may be an included in the other figures. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

### DETAILED DESCRIPTION OF THE ILLUSTRATED EMBODIMENTS

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

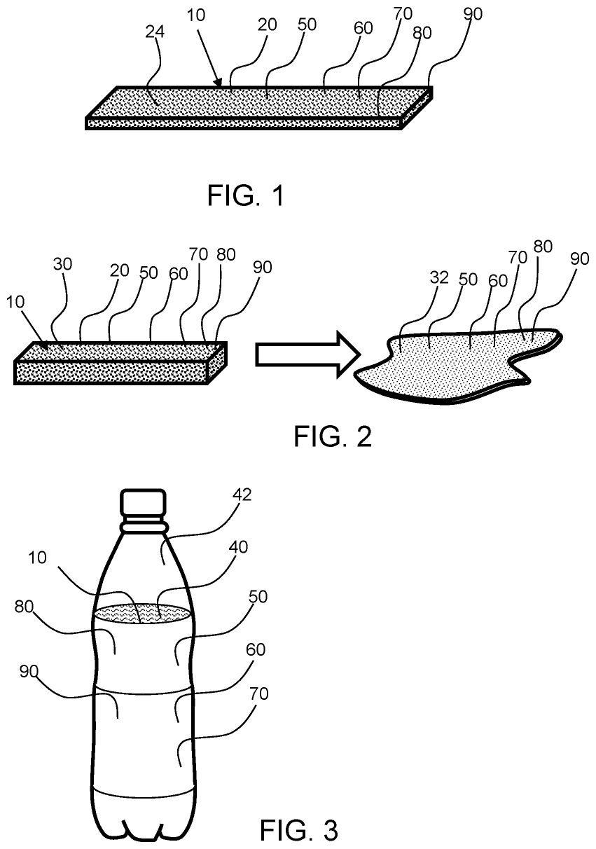

As shown in FIG. 1, an exemplary oral delivery vehicle 10 is a tablet 20 in the form of a stick of chewing gum 24. The chewing gum comprises the nicotinamide adenine dinucleotide 50 and may also include other ameliorating therapeutic ingredients such as a stimulate 60, such as caffeine, cannabidiol 70, a vitamin 80, such as a B vitamin, B6, or B12 for example, and a salt or electrolyte 90.

As shown in FIG. 2, an exemplary oral delivery vehicle 10 is a tablet 20 in the form of a soluble tablet 30 that is shown also as a dissolved tablet 32. The soluble tablet, like the stick of chewing gum is a solid but dissolves when inserted into the mouth. The soluble tablet may include gelatin, mucilage, or gum arabic. The composition may be configured to ensure the soluble table remains a solid over a delivery time, such as about one minute or more, about two minutes or more or even about five minutes or more. The soluble tablet comprises the nicotinamide adenine dinucleotide 50 and may also include other ameliorating therapeutic ingredients such as a stimulate 60, such as caffeine, cannabidiol 70, a vitamin 80, such as a B vitamin, B6, or B12 for example, and a salt or electrolyte 90.

As shown in FIG. 3, exemplary oral delivery vehicle 10 is a beverage 40 in a beverage container 42. As described herein, the beverage may be placed in the mouth and retained in the mouth for a delivery time to effectively enable the NAD to absorb through blood vessels in the mouth and into the bloodstream. After a delivery time, the beverage may be discarded or swallowed. This process may be repeated a number of times to increase the NAD concentration. The beverage comprises the nicotinamide adenine dinucleotide 50 and may also include other ameliorating therapeutic ingredients such as a stimulate 60, such as caffeine, cannabidiol 70, a vitamin 80, such as a B vitamin, B6, or B12 for example, and a salt or electrolyte 90.

It will be apparent to those skilled in the art that various modifications, combinations and variations can be made in the present invention without departing from the scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

What is claimed:

1. An oral method of administering nicotinamide adenine dinucleotide (NAD) to an individual, comprising:
a) providing an oral delivery vehicle comprising nicotinamide adenine dinucleotide; and
b) placing said oral delivery vehicle into mouth of said individual for a delivery time that is effective for absorption of said nicotinamide adenine dinucleotide into blood vessels in the mouth.

2. The method of claim 1, wherein the nicotinamide adenine dinucleotide is provided in the oral delivery vehicle in a concentration of at least 10 mg.

3. The method of claim 1, wherein the nicotinamide adenine dinucleotide is provided in the oral delivery vehicle in a concentration of at least 20 mg.

4. The method of claim 1, wherein the delivery time is at least two second.

5. The method of claim 1, wherein the delivery time is at least 30 seconds.

6. The method of claim 1, wherein the delivery time is at least two minutes.

7. The method of claim 1, wherein the oral delivery vehicle further comprises caffeine.

8. The method of claim 1, wherein the oral delivery vehicle comprises a stimulant and wherein a ratio of nicotinamide adenine dinucleotide to stimulant is between 0.5:1 and 1.5:1.

9. The method of claim 8, wherein the stimulant is caffeine.

10. The method of claim 1, wherein the oral delivery vehicle further comprises cannabidiol.

11. The method of claim 1, wherein the cannabidiol in included in a concentration of at least 5 mg.

12. The method of claim 11, wherein the oral delivery vehicle comprises a stimulant and wherein a ratio of nicotinamide adenine dinucleotide to stimulant is between 0.5:1 and 1.5:1.

13. The method of claim 12, wherein the stimulant is caffeine.

14. The method of claim 1, wherein the oral delivery vehicle is a tablet.

15. The method of claim 14, wherein the tablet is a soluble tablet.

16. The method of claim 15, wherein the method comprises placing the soluble tablet under a tongue for said delivery time.

17. The method of claim 16, wherein the delivery time is at least 30 seconds.

18. The method of claim 14, wherein the tablet is chewing gum.

19. The method of claim 18, further comprising chewing on said chewing gum for said delivery time.

20. The method of claim 1, wherein the oral delivery vehicle is a beverage.

21. The method of claim 20, further comprising swallowing the beverage after the delivery time.

22. The method of claim 20, further comprising repeating placing said oral delivery vehicle into mouth of said individual for a delivery time that is effective for absorption of said nicotinamide adenine dinucleotide into blood vessels in the mouth.

23. The method of claim 22, further comprising swallowing the beverage after the delivery time.

24. The method of claim 20, wherein oral delivery vehicle comprises an ameliorating ingredient selected from the group consisting of a vitamin, an electrolyte and an amino acid.

25. The method of claim 24, wherein a portion of the ameliorating ingredient is absorbed into the individual's bloodstream through the individual's gastrointestinal tract.

26. The method of claim 1, wherein the oral delivery vehicle further comprises an ameliorating ingredient comprising a vitamin.

27. The method of claim 1, wherein the oral delivery vehicle further comprises an ameliorating ingredient comprising an electrolyte

28. The method of claim 1, wherein the oral delivery vehicle further comprises an ameliorating ingredient comprising an amino acid.

29. The method of claims 26 to 28, wherein a portion of the ameliorating ingredient is absorbed into the individual's bloodstream through the individual's gastrointestinal tract.
